(19) United States
(12) Patent Application Publication
(10) Pub. No.: US 2024/0062673 A1
(43) Pub. Date: Feb. 22, 2024

(54) RELAXATION SUPPORT APPARATUS, RELAXATION SUPPORT METHOD AND PROGRAM

(71) Applicant: NIPPON TELEGRAPH AND TELEPHONE CORPORATION, Tokyo (JP)

(72) Inventors: Arinobu NIIJIMA, Tokyo (JP); Toki TAKEDA, Tokyo (JP); Ryosuke AOKI, Tokyo (JP); Yukio KOIKE, Tokyo (JP)

(21) Appl. No.: 18/259,347

(22) PCT Filed: Jan. 14, 2021
(86) PCT No.: PCT/JP2021/001110

§ 371 Date: Jun. 26, 2023

(51) Int. Cl. G09B 15/06; A61N 1/36; A61N 1/04; A61N 1/02

(52) U.S. Cl. CPC G09B 15/06; A61N 1/36003; A61N 1/36031; A61N 1/0452; A61N 1/0476; A61N 1/025

(57) ABSTRACT

A relaxation support device including: an electrical stimulation control unit configured to control electrical stimulation to a muscle different from a muscle to be relaxed based on a muscle activity of the muscle to be relaxed; and an electrical stimulation presentation unit configured to present the electrical stimulation to a muscle different from the muscle to be relaxed.

### TECHNICAL FIELD

The present invention relates to a technique for relaxation of muscles.

### BACKGROUND ART

In practice of playing a musical instrument, it is possible to support a user's musical performance learning by using tactile feedback applying electrical muscle stimulation (EMS).

For example, in Non Patent Literature 1, a player is notified of which finger to move by EMS in order to learn finger movement in playing a Japanese harp. In addition, in Non Patent Literature 2, in order to learn a sense of rhythm in playing a drum, a beat rhythm is transmitted to a player by EMS.

### CITATION LIST

### Non Patent Literature

Non Patent Literature 1: Emi Tamaki, Takashi Miyaki, and Jun Rekimoto. 2011. PossessedHand: techniques for controlling human hands using electrical muscles stimuli. In Proceedings of the SIGCHI Conference on Human Factors in Computing Systems (CHI '11). 543-552.
Non Patent Literature 2: Ayaka Ebisu, Satoshi Hashizume, Kenta Suzuki, Akira Ishii, Mose Sakashita, and Yoichi Ochiai. 2017. Stimulated percussions: method to control human for learning music by using electrical muscle stimulation. In Proceedings of the 8th Augmented Human International Conference (AH '17). 1-5.

### SUMMARY OF INVENTION

### Technical Problem

In playing a musical instrument, not only movement of fingers and a sense of rhythm, but also playing without applying a force to fingers is important. For example, if a player applies force to fingers when striking keys quickly and continuously like during a piano tremolo performance, the fingers become tired easily and may lead to injury such as tenosynovitis.

In a case where EMS is used to support the player so that the player can perform in a relaxed manner, there is a problem that muscles contract and thus the fingers cannot be relaxed when EMS is presented, by applying a conventional use of EMS, to muscles (extensor pollicis longus, flexor longus, extensor digitorum communis, flexor digitorum superficialis, and the like) that move the fingers.

The present invention has been made in view of the above points, and it is an object of the present invention to provide a technique capable of relaxing a specific muscle.

### Solution to Problem

According to the disclosed technique, there is provided a relaxation support device including:
an electrical stimulation control unit configured to control electrical stimulation to a muscle different from a muscle to be relaxed based on a muscle activity of the muscle to be relaxed; and
an electrical stimulation presentation unit configured to present the electrical stimulation to a muscle different from the muscle to be relaxed.

### Advantageous Effects of Invention

According to the disclosed technology, there is provided a technique that makes it possible to relax a specific muscle.

### BRIEF DESCRIPTION OF DRAWINGS

FIG. 1 is a configuration diagram of a relaxation support device according to one embodiment of the present invention.

FIG. 2 is a flowchart for explaining an operation example of the relaxation support device.

FIG. 3 is a configuration diagram of a relaxation support device according to one embodiment of the present invention.

FIG. 4 is a configuration diagram of an electrical stimulation control apparatus.

FIG. 5 is a diagram illustrating an example of a relaxation support device.

FIG. 6 is a diagram illustrating an example of a relaxation support device.

FIG. 7 is a diagram illustrating a hardware configuration example of devices.

FIG. 8 is a flowchart for explaining an operation example of the relaxation support device.

FIG. 9 is a diagram illustrating average % MVC during each performance before and after an EMS presentation.

### DESCRIPTION OF EMBODIMENTS

The following describes one embodiment of the present invention (the present embodiment) with reference to the drawings. The embodiment to be described below is merely an example, and embodiments to which the present invention is applied are not limited to the following embodiment.

In the examples described below, a muscle to be relaxed is a muscle that moves a finger, and a muscle to which electrical stimulation is presented is a muscle that moves a wrist. However, these are examples in a case where exercise such as the tremolo performance is assumed. The present invention is not limited to these examples, and can be applied to assist relaxation of muscles involved in various exercises.

### Overview of Present Embodiment

In the present embodiment, electrical stimulation is presented to a muscle of a site different from a muscle to be relaxed, which moves the muscle of the different site, thereby reducing tension in the muscle to be relaxed. For example, in the tremolo performance, by presenting the electrical stimulation to muscles (supinator muscle, supinator muscle, and the like) that rotate the wrist, which are muscles of a region different from the muscles that move fingers, the wrist is rotated to play the tremolo without applying force to the fingers, so that the tension in the muscles that move the fingers can be reduced.

### (Device Configuration and Operation)

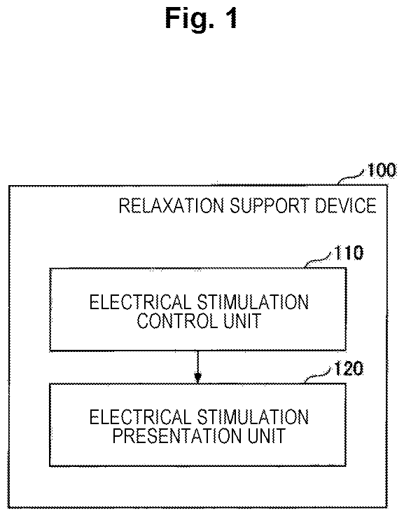

FIG. 1 illustrates a configuration example of a relaxation support device 100 according to the present embodiment. As illustrated in FIG. 1, the relaxation support device 100 includes an electrical stimulation control unit 110 that controls an electrical stimulation presentation parameter, and an electrical stimulation presentation unit 120 that presents electrical stimulation to a specific site.

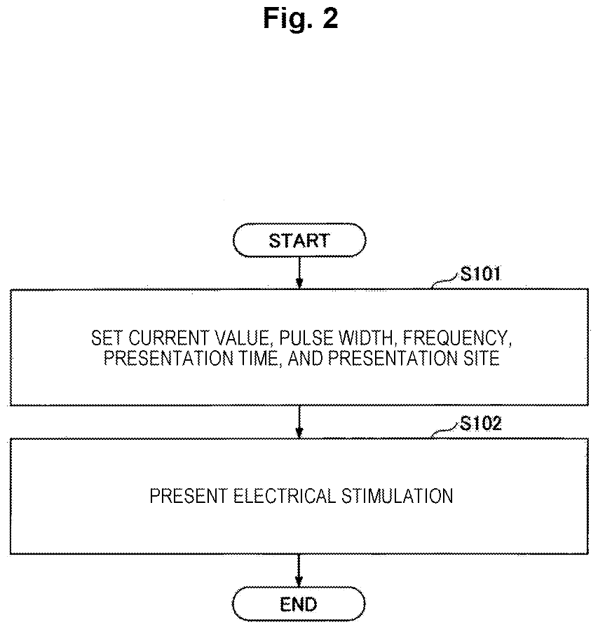

FIG. 2 is a flowchart illustrating an operation procedure of the relaxation support device 100. As illustrated in FIG. 2, in S101, the electrical stimulation control unit 110 sets a current value, a pulse width, a frequency, a presentation time, a presentation site, and the like as parameters of the electrical stimulation.

In S102, the electrical stimulation presentation unit 120 presents the electrical stimulation to the muscle through electrodes attached to a skin surface based on the parameters set by the electrical stimulation control unit 110.

### EXAMPLES

More specific examples will be described below.

### <Device Configuration>

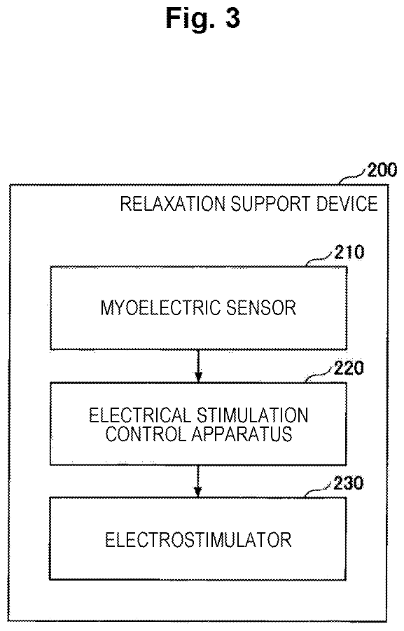

FIG. 3 is a configuration diagram of a relaxation support device 200 that presents the electrical stimulation according to tension in a finger. As illustrated in FIG. 3, the relaxation support device 200 includes a myoelectric sensor 210, an electrical stimulation control apparatus 220, and an electrostimulator 230. The relaxation support device 200 may be referred to as a relaxation support system.

The myoelectric sensor 210 is a myoelectric sensor attached to the skin surface of the site where a muscle for moving a finger to be relaxed is located. The electrical stimulation control apparatus 220 is connected to the myoelectric sensor 210, and determines presence or absence of tension in the muscle that moves the finger based on values of measurement result from the myoelectric sensor 210. The electrostimulator 230 is the electrodes attached to, for example, the skin surface of the site where the muscles for moving the wrist (for example, pronator muscle and the supinator muscle) are located, at a different site from the muscle for moving the finger. The electrodes are connected to the electrical stimulation control apparatus 220, and present electrical stimulation to the muscles based on an electrical signal from the electrical stimulation control apparatus 220.

Note that “the myoelectric sensor 210+the electrical stimulation control apparatus 220” correspond to the electrical stimulation control unit 110 illustrated in FIG. 1. It is also possible to adopt a configuration not including the myoelectric sensor 210, and in this case, the electrical stimulation control apparatus 220 corresponds to the electrical stimulation control unit 110 illustrated in FIG. 1. The electrostimulator 230 corresponds to the electrical stimulation presentation unit 120 illustrated in FIG. 1.

The myoelectric sensor 210, the electrical stimulation control apparatus 220, and the electrostimulator 230 may be referred to as a muscle activity measurement unit, an electrical stimulation control unit, and an electrical stimulation presentation unit, respectively.

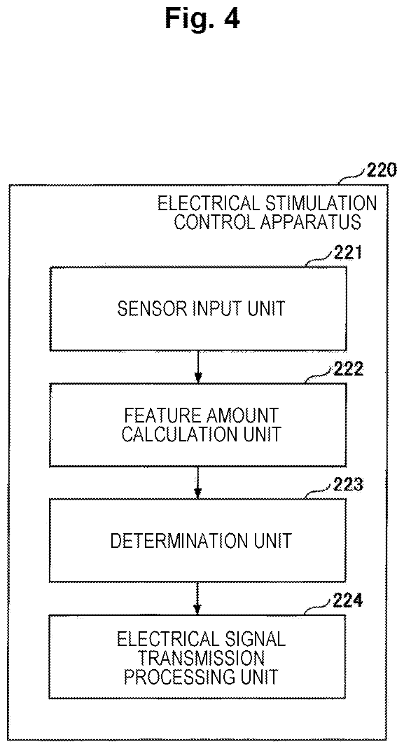

FIG. 4 is a functional configuration diagram of the electrical stimulation control apparatus 220. As illustrated in FIG. 4, the electrical stimulation control apparatus 220 includes a sensor input unit 221, a feature amount calculation unit 222, a determination unit 223, and an electrical signal transmission processing unit 224.

The sensor input unit 221 inputs the values of the measurement result received from the myoelectric sensor 210, and passes the values to the feature amount calculation unit 222.

The feature amount calculation unit 222 calculates a feature amount from the values of the myoelectric sensor 210, and passes the calculated feature amount to the determination unit 223. The determination unit 223 compares the feature amount with a threshold to determine whether or not there is tension in the muscle that moves the finger, and passes a determination result to the electrical signal transmission processing unit 224.

Upon receiving the determination result that there is tension, the electrical signal transmission processing unit 224 sets a current value, a pulse width, a frequency, a presentation time, a presentation site, and the like, and transmits an electrical signal to the electrostimulator 230 based on the set values, thereby causing the electrostimulator 230 to present the electrical stimulation.

Regarding a method for selecting a site for presenting the electrical stimulation in the present embodiment, a selection may be made based on existing ergonomic knowledge, for example. In addition, myoelectric potentials of a plurality of muscles may be measured in advance, and one muscle may be selected from among muscles linked with a certain operation (for example, extensor digitorum communis, the supinator muscle, and biceps brachii muscle in the tremolo performance).

For selection of the muscle (selection of the presentation site), the selected muscle (specifically, the electrodes that provide electrical stimulation to the selected muscle) may be set in advance in the electrical signal transmission processing unit 224, or the electrical signal transmission processing unit 224 may determine a type of the operation from a fluctuation in the values of a muscle strength sensor 221 and select the muscle (that is, the electrodes) suitable for the operation.

Furthermore, for the current value, the pulse width, the frequency, and the presentation time, for example, appropriate values may be determined by experiment or the like, and the values may be set in the electrical signal transmission processing unit 224, and the electrical signal transmission processing unit 224 may use the set values. In addition, a plurality of sets of “current value, pulse width, frequency, and presentation time” according to the operation may be set in the electrical signal transmission processing unit 224, and the electrical signal transmission processing unit 224 may determine the type of the operation from the fluctuation in the values of the muscle strength sensor 221 and the like, and select the “current value, pulse width, frequency, and presentation time” suitable for the operation.

The electrodes for presenting electrical stimulation constituting the electrostimulator 230 may be disposable electrodes, cloth electrodes, or other electrodes.

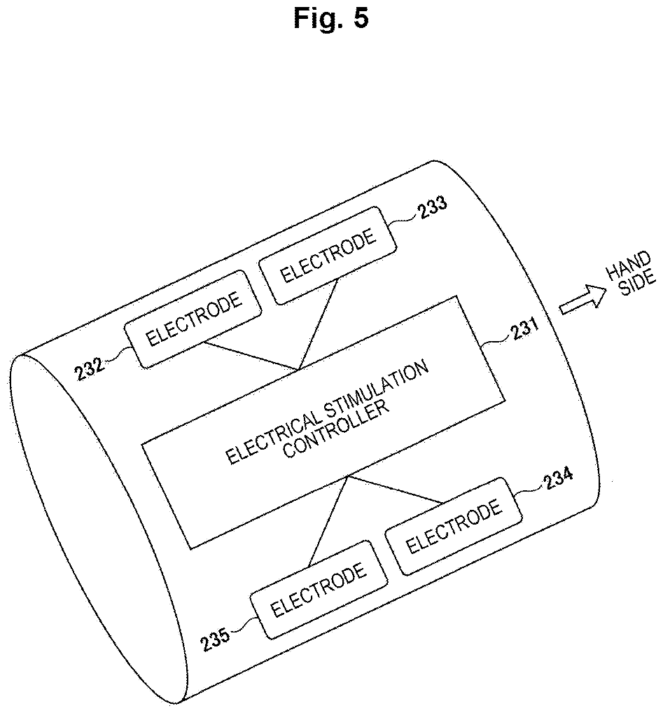

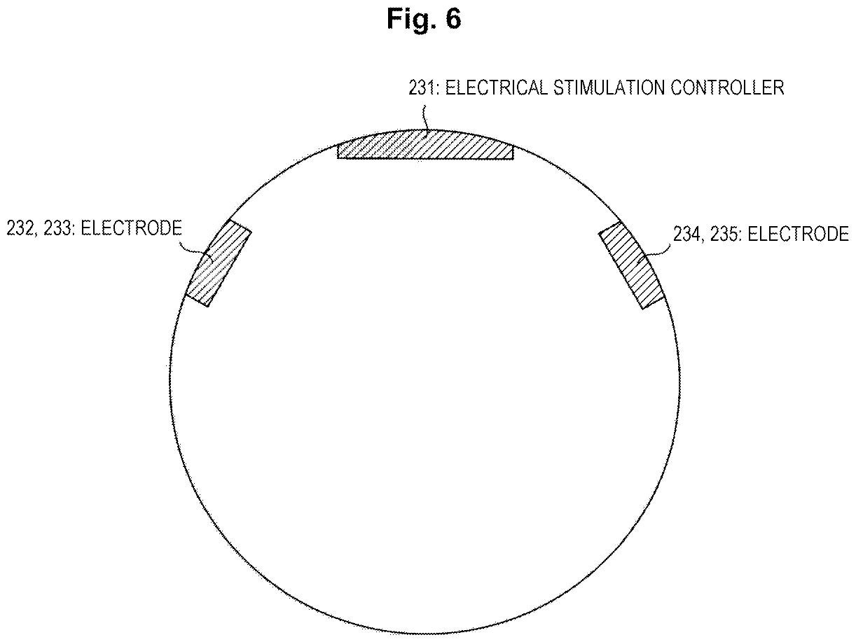

Furthermore, the relaxation support device 200 may be an elbow supporter type device in which two sets of cloth electrodes are arranged diagonally. FIG. 5 illustrates an example of the relaxation support device 200 in which cloth electrodes 232, 233, 235, and 234 are disposed diagonally inside the elbow supporter. FIG. 6 illustrates a cross-sectional view when viewed from a hand side. The electrical stimulation controller 231 corresponds to the electrical stimulation control apparatus 220. The electrical stimulation controller 231 may be provided with the myoelectric sensor.

When the relaxation support device 200 is an elbow supporter type device in which two sets of cloth electrodes are arranged diagonally, it is easy to attach and detach, and it is possible to present the electrical stimulation to the supinator muscle and the pronator muscle. As shown in experimental results to be described later, by presenting the electrical stimulation to the pronator muscle and the supinator muscle, it is possible to reduce the muscle activity of the muscles that move fingers (for example, the extensor pollicis longus and the extensor digitorum communis), and it is possible to support the performance while reducing the tension in the finger.

### <Hardware Configuration Example>

The electrical stimulation control apparatus 220 may be realized by causing a computer (for example, a PC) to execute a program, or may be realized by dedicated hardware such as an integrated circuit (IC).

In a case where the electrical stimulation control apparatus 220 is implemented by causing a computer to execute a program, the program can be recorded, stored, or distributed on a computer-readable recording medium (a portable memory or the like). Furthermore, the program can also be provided through a network such as the Internet or an electronic mail.

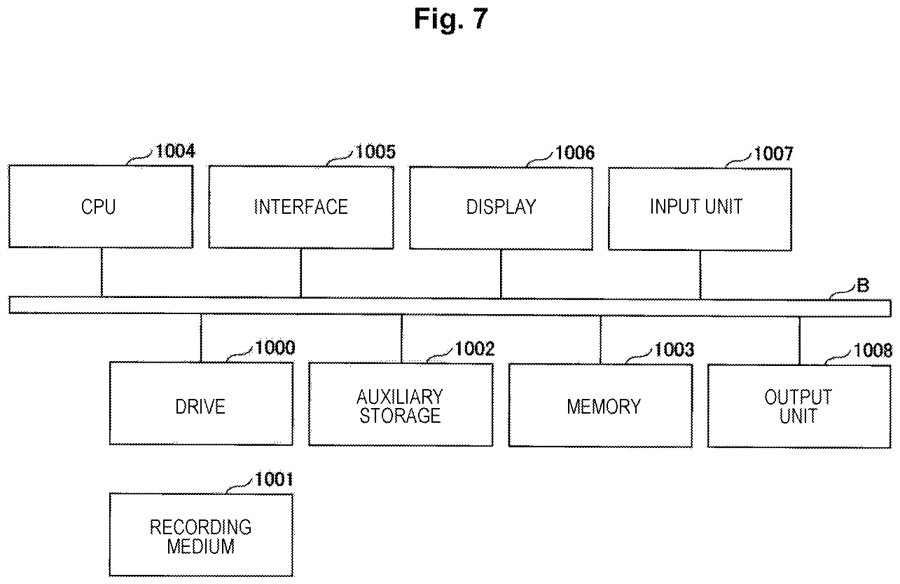

FIG. 7 is a diagram illustrating a hardware configuration example of the above computer. The computer in FIG. 7 includes a drive 1000, an auxiliary storage 1002, a memory 1003, a central processing unit (CPU) 1004, an interface 1005, a display 1006, an input unit 1007, an output unit 1008, and the like that are connected to each other by a bus B.

The program for implementing the processing in the computer is provided by, for example, a recording medium 1001 such as a CD-ROM or a memory card. When the recording medium 1001 that stores the program is set in the drive 1000, the program is installed from the recording medium 1001 to the auxiliary storage 1002 via the drive 1000. However, the program is not necessarily installed from the recording medium 1001, and may be downloaded from another computer via the network. The auxiliary storage 1002 stores the installed program and also stores necessary files, data, and the like.

In a case where an instruction to start the program is made, the memory 1003 reads and stores the program from the auxiliary storage 1002. The CPU 1004 implements a function related to the electrical stimulation control apparatus 220 in accordance with the program stored in the memory 1003. The interface 1005 is used as the interface for connecting to an external device. For example, the myoelectric sensor 210 and the electrostimulator 230 may be connected via the interface 1005. The display 1006 displays a graphical user interface (GUI) or the like by the program. The input unit 1007 includes a keyboard and a mouse, buttons, a touch panel, or the like, and is used to input various operation instructions. The output unit 1008 outputs a calculation result.

### <Operation Example>

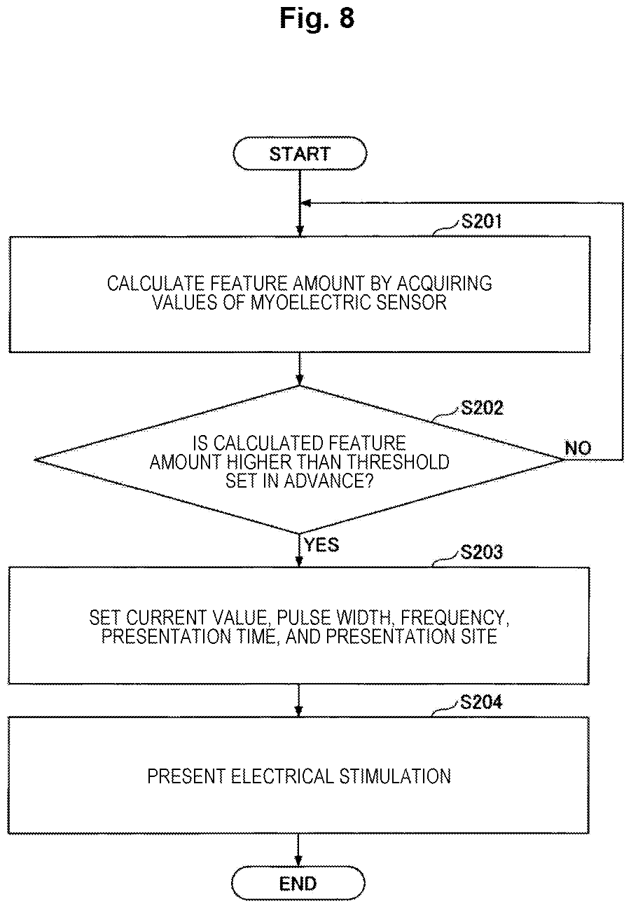

Next, the operation example of the relaxation support device 200 will be described with reference to a flowchart of FIG. 8. In S201, the electrical stimulation control apparatus 220 receives the values of the measurement result from the myoelectric sensor 210, and calculates the feature amount for determining the tension in the muscle that moves the finger.

The feature amount to be calculated is not limited to a specific one, but for example, a root mean square value every 200 milliseconds is calculated as the feature amount.

In S202, the electrical stimulation control apparatus 220 compares the calculated feature amount with a preset threshold, and in a case where the calculated feature amount is higher than the preset threshold, it is determined that there is tension in the muscle that moves the finger, and the processing proceeds to S203.

In S203, the electrical stimulation control apparatus 220 sets the current value, the pulse width, the frequency, the presentation time, the presentation site, and the like, and in S204, the electrical signal is transmitted to the electrostimulator 230 based on the set values. The electrostimulator 230 has the electrodes attached to the skin surface of the site where the muscle that moves the wrist is located, and presents the electrical stimulation to the muscle through the electrode.

### Experimental Results

Using the relaxation support device according to the present invention, an experiment was conducted to verify whether the muscles of fingers are relaxed in the piano tremolo performance, and the experimental results thereof will be described.

Experiments were conducted with eight subjects who were piano players of a beginner level. A tremolo performance task was performed for a certain period of time in which a subject played C notes one octave apart with the thumb and the little finger at a constant tempo. In the middle of the experiment, EMS was alternately presented to the pronator muscle and the supinator muscle to cause an operation of rotating the wrist. Then, the same tremolo was played again without stimulation. At this time, it was verified whether there was a difference in the muscle activity before and after the EMS was presented. The measured myoelectric potentials were two kinds of myoelectric potentials of the extensor pollicis longus and the extensor digitorum communis, and the muscle activity during the performance was estimated using an index of the percentage of maximum voluntary contraction (% MVC). The % MVC is an index indicating a degree of muscle activity.

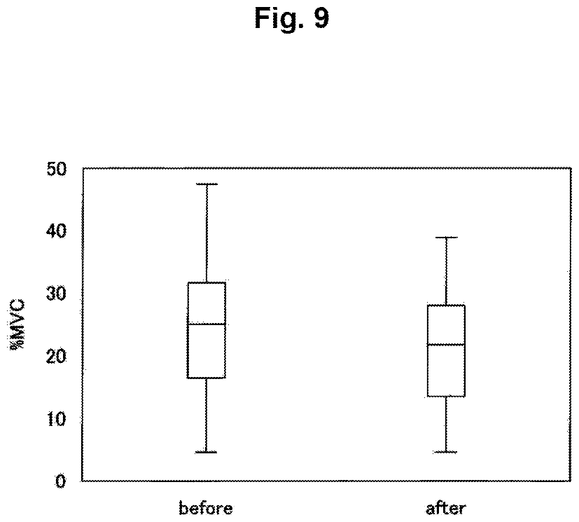

FIG. 9 illustrates average values of % MVC during the performance before and after the EMS presentation. From the experimental results, the value of % MVC significantly decreased after the EMS presentation. Therefore, it can be said that the muscle activity of the muscles that move the fingers can be reduced by teaching a rotation operation of the wrist by EMS. In other words, it can be said that the EMS assisted in the performance while reducing the tension in the fingers.

### Effects of Embodiment

According to the relaxation support device of the present embodiment, by presenting the electrical stimulation to a muscle different from the muscle of the finger, which is a muscle to be relaxed, it is possible to play without applying force to the finger, so that fatigue is less likely to occur, leading to prevention of injury.

### Summary of Embodiment

The present description discloses a relaxation support device, a relaxation support method, and a program according to at least the following items.
(Item 1)
   A relaxation support device including:
   an electrical stimulation control unit configured to control electrical stimulation to a muscle different from a muscle to be relaxed based on a muscle activity of the muscle to be relaxed; and
   an electrical stimulation presentation unit configured to present the electrical stimulation to a muscle different from the muscle to be relaxed.
(Item 2)
   The relaxation support device according to item 1 in which the electrical stimulation presentation unit presents the electrical stimulation to a muscle linked with the muscle to be relaxed in a certain exercise.
(Item 3)
   The relaxation support device according to item 1 or 2, further including
   a muscle activity measurement unit configured to measure a muscle activity of the muscle to be relaxed in which
   the electrical stimulation control unit controls the electrical stimulation according to a magnitude of the muscle activity measured by the muscle activity measurement unit, and
   the electrical stimulation presentation unit presents the electrical stimulation to a site different from a site measured by the muscle activity measurement unit.
(Item 4)
   The relaxation support device according to any one of items 1 to 3 in which in a form of an elbow supporter, two sets of electrodes are arranged diagonally.
(Item 5)
   A relaxation support method that is executed by a relaxation support device, the relaxation support method including:
   an electrical stimulation control step of controlling electrical stimulation to a muscle different from a muscle to be relaxed based on a muscle activity of the muscle to be relaxed; and
   an electrical stimulation presentation step of presenting the electrical stimulation to a muscle different from the muscle to be relaxed.
(Item 6)
   A program for causing a computer to function as the electrical stimulation control unit in the relaxation support device according to any one of items 1 to 4.

Although the present embodiment has been described above, the present invention is not limited to such a specific embodiment, and various modifications and changes can be made within the scope of the gist of the present invention described in the claims.

### REFERENCE SIGNS LIST

100, 200 Relaxation support device
110 Electrical stimulation control unit
120 Electrical stimulation presentation unit
210 Myoelectric sensor
220 Electrical stimulation control apparatus
230 Electrostimulator
221 Sensor input unit
222 Feature amount calculation unit
223 Determination unit
224 Electrical signal transmission processing unit
232, 233, 235, 234 Electrode
231 Electrical stimulation controller
1000 Drive
1001 Recording medium
1002 Auxiliary storage
1003 Memory
1004 CPU
1005 Interface
1006 Display
1007 Input unit

1. A relaxation support device comprising:
a processor; and
a memory storing program instructions that cause the processor to:
control electrical stimulation to a muscle different from a muscle to be relaxed based on a muscle activity of the muscle to be relaxed; and
present the electrical stimulation to the muscle different from the muscle to be relaxed.

2. The relaxation support device according to claim 1, wherein the processor is configured to present the electrical stimulation to a muscle linked with the muscle to be relaxed in a certain exercise.

3. The relaxation support device according to claim 1, wherein the processor is further configured to
measure a muscle activity of the muscle to be relaxed,
control the electrical stimulation according to a magnitude of the muscle activity, and
present the electrical stimulation to a site different from a site where the muscle activity is measured.

4. The relaxation support device according to claim 1, wherein in a form of an elbow supporter, two sets of electrodes are arranged diagonally.

5. A relaxation support method that is executed by a relaxation support device, the relaxation support method comprising:
controlling electrical stimulation to a muscle different from a muscle to be relaxed based on a muscle activity of the muscle to be relaxed; and
presenting the electrical stimulation to the muscle different from the muscle to be relaxed.

6. A non-transitory computer-readable recording medium storing a program for causing a computer to function as the relaxation support device according to claim 1.
